(19) United States
(12) Patent Application Publication
(10) Pub. No.: US 2011/0065749 A1
(43) Pub. Date: Mar. 17, 2011

(54) METHOD AND PHARMACEUTICAL COMPOSITION FOR TREATMENT OF MENTAL DISORDERS

(75) Inventor: Kazuo Sakai, Tokyo (JP)

(73) Assignee: ACTIVE CO. LTD., Tokyo (JP)

(21) Appl. No.: 12/992,681

(22) PCT Filed: Jun. 2, 2009
(86) PCT No.: PCT/JP2009/002455

§ 371 Date: Nov. 15, 2010

(30) Foreign Application Priority Data
Jun. 4, 2008 (JP) 2008-146755

(51) Int. Cl. A61K 31/445; A61K 31/4453; A61P 25/24; A61P 25/18; A61P 25/22; A61P 25/28; A61P 25/32

(52) U.S. Cl. 514/319; 514/317

(57) ABSTRACT

A method of treating a mental disorder including administering to a patient in need thereof, an effective amount of a pharmaceutical composition including 1,1-diphenyl-4-piperidine-1-ylbuthan-1-ol or a pharmaceutically acceptable salt thereof. The mental disorder is depression, bipolar disorder, anxiety disorder, impulsive disorder, bulimia, panic disorder, social anxiety disorder, insomnia, attention deficit hyperactivity disorder (ADHD), schizophrenia, dementia, personality disorder, alcoholism, dissociative disorder, sleep apnea syndrome, or fibromyalgia. In a preferred embodiment, the mental disorder as a therapeutic target is fibromyalgia, chronic fatigue syndrome (CFS), or depression with pain, and the administration of the pharmaceutical composition is capable of reducing the pain caused by these diseases.

### TECHNICAL FIELD

The present invention relates to methods and pharmaceutical compositions to treat mental disorders, and more specifically, to methods and pharmaceutical compositions to treat depression, bipolar disorder, anxiety disorder, impulsive disorder, bulimia, panic disorder, social anxiety disorder, insomnia, attention deficit hyperactivity disorder (ADHD), schizophrenia, dementia, personality disorder, alcoholism, dissociative disorder, sleep apnea syndrome, or fibromyalgia. In addition, the methods and pharmaceutical compositions of the present invention can be used to improve insomnia or delirium associated with dementia, or behavioral and psychological symptoms of dementia such as persecutory delusion, prowl, and violent behavior.

### BACKGROUND ART

It has been estimated that approximately 4% of the people in the world suffer from depression. Depression differs from any previously known neurological diseases, and affects people in all walks of society, from the very young to the very old. It often occurs without the presence of a precipitating event, and can be unresponsive to psychotherapy, environmental changes or to pharmacotherapy with the currently available medications.

When an individual is suffering from depression, he or she will usually be in a depressed mood, as well as experience a loss of interest or pleasure in all, or almost all, activities. These and associated symptoms last for a period of at least two weeks. The associated symptoms may include, but not be limited to, appetite disturbance, change in weight, sleep disturbance, psychomotor agitation or retardation, decreased energy, feelings of worthlessness or excessive or inappropriate guilt, difficulty thinking or concentrating, and recurrent thoughts of death or suicidal ideation or attempts. Further, a person suffering from depression can also experience fearfulness, anxiety, irritability, brooding or obsessive rumination, excessive concern with physical health, panic attacks, and phobias.

For drug therapy for depression, there are several types of drugs such as tricyclic antidepressants, selective serotonin reuptake inhibitors (SSRIs), monoamine oxidase (MAO) inhibitors, psychoactive drugs, and other antidepressants. Almost all antidepressants need to be taken regularly for at least several weeks before they start to exert their effectiveness. The probability that a single antidepressant is effective to an individual is likely to be about 65%, and thus, if a single drug is not effective on depression, several antidepressants are used in combination.

Selective serotonin reuptake inhibitors (SSRIs) are currently the most commonly used antidepressants. SSRIs are effective on depressed mood, dysthymia, and also other mental disorders that frequently occur with depression. Nausea, diarrhea, trembling, weight loss, and headache are actually the side effects of SSRIs, but the symptoms are generally mild and disappear during consecutive use. Long-term administration of SSRIs, however, may cause other side effects such as body weight gain. Some of SSRIs may also cause withdrawal syndromes such as dizziness, anxiety, excitation, and flu-like symptoms, upon sudden withdrawal.

Difenidol hydrochloride (1,1-diphenyl-4-piperidine-1-ylbuthan-1-ol monohydrochloride) is an effective ingredient of a medicine that is used for dizziness attributed to inner ear disorder. It was found, by dog experiments, to remit the spasm of the vertebral artery, and increase the blood flow thereof (see, for example, Non-Patent Document 1). It inhibited the evoked potential of hypothalamus by vestibular nuclei stimulation by rat experiments (see, for example, Non-Patent Document 2). Rabbit experiments also showed that it inhibited the emergence of nystagmus by experimental disorder of balance function (see, for example, Non-Patent Document 3). Based on these pharmacological effects, difenidol hydrochloride is used for the treatment of dizziness; however, its pharmacological effect on mental disorders has not been known.

### CITATION LIST

### Non-Patent Documents

Non-Patent Document 1: HIKIDA, Hideaki et al., Gendai no Rinsho, Vol. 5, p. 471, 1971

Non-Patent Document 2: MATSUNAGA, Toru et al., Practica Oto-Rhino-Laryngologica, Vol. 66, p. 883, 1973

Non-Patent Document 3: MATSUNAGA, Toru et al., Practica Oto-Rhino-Laryngologica, Vol. 64, p. 1095, 1971

### SUMMARY OF INVENTION

### Technical Problem

A variety of drugs have been developed for the treatment of depression, but the efficacy of a drug often depends on each individual patient. There is also refractory depression, which is resistant to most of the drugs. Antianxiety drugs are used for the treatment of anxiety disorder, but they often cause dependence, resistance, staggering gait as a side effect, and memory retention disorder. The drugs cannot be taken with alcohol, and often cause an overdose accident. Antiepileptic drugs are used for improving impulsivity, excited state, and manic state, but with many side effects. No effective drugs have been found for ADHD. In view of such a situation, it is an object of the present invention to provide a therapeutic drug that has less side effects than conventional drugs, or a novel adjunctive drug that is capable of drastically reducing the dose of existing drugs.

### Solution to Problem

The present inventor has found a novel method and pharmaceutical composition that can greatly improve the treatment of various mental disorders such as depression, during the course of his daily clinical practice. In the brain, oxygen consumption of hypoactive regions becomes lower. This is due to the decreased blood flow of the regions, which causes further dysfunction of the brain, and leads to the vicious circle of lower blood flow. In this situation, an improvement or repair of the hypoactive regions is difficult due to the insufficient blood flow. Thus, if the blood flow of the hypoactive regions were improved, the original self-repair ability of the patient would be exerted maximally. In the case of pharmaceutical therapy of mental disorders using conventional drugs, it would take a lot of time to repair the neural circuits with the lowered blood flow. In view of this, a concomitant use of a drug that can increase the blood flow of the hypoactive regions would enhance the efficacy of the conventional drugs, and shorten the time for the conventional drugs to start to work.

It has been confirmed that depression develops from dysfunction of various unspecified regions in the brain. In the case of depression, considering that dysfunction of the neural circuits will lead to the decrease of blood flow, the self-regenerative power of the brain cannot be elevated without supply of much more oxygen to the tissue, regardless of whether it is a cause or effect. Therefore, it is reasonably understood that an increase of blood flow in the brain is effective in ameliorating depression. The inventor has then discovered that a therapeutic drug for dizziness, which is commercially available as a trade name Cephadol®, acts to improve the blood flow in deep regions of the brain. The inventor has further found that this action is also effective on various mental disorders such as anxiety, impulsive disorder and alcoholism, as well as depression, and thereby completed the present invention.

Accordingly, a pharmaceutical composition for use in treating mental disorders of the present invention comprises 1,1-diphenyl-4-piperidine-1-ylbuthan-1-ol or a pharmaceutically acceptable salt thereof. The mental disorders include, but are not limited to, depression, bipolar disorder, anxiety disorder, impulsive disorder, bulimia, panic disorder, social anxiety disorder, insomnia, attention deficit hyperactivity disorder (ADHD), schizophrenia, dementia, personality disorder, alcoholism, dissociative disorder, sleep apnea syndrome, and fibromyalgia. The pharmaceutical composition of the present invention can be used alone or in combination with another antidepressant, antianxiety, antipsychotic, antiepileptic, or hypnotic drug. In a preferred embodiment, the pharmaceutical composition of the present invention can be administered simultaneously with another antidepressant, antianxiety, antipsychotic, antiepileptic, or hypnotic drug, or can be administered alone after discontinuation of treatment with the other antidepressant, antianxiety, antipsychotic, antiepileptic, or hypnotic drug.

In another aspect of the present invention, there is provided a pharmaceutical composition for improving insomnia or delirium associated with dementia, or behavioral and psychological symptoms of dementia such as persecutory delusion, prowl, and violent behavior, the pharmaceutical composition comprising 1,1-diphenyl-4-piperidine-1-ylbuthan-1-ol or a pharmaceutically acceptable salt thereof. In a preferred embodiment, the pharmaceutical composition is prepared as a drug mixture with an antidementia drug, more preferably with donepezil hydrochloride, for concurrent administration.

### EFFECT OF THE INVENTION

The pharmaceutical composition of the present invention shows an antidepressive activity by itself, and also enhances the effects of other antidepressants. Its effect appears so fast as to be found on the next day of administration. Thus, the pharmaceutical composition of the present invention starts to work significantly faster than conventional antidepressants which start to work in, e.g., 2 to 6 weeks. In addition, it should be emphasized that the pharmaceutical composition has few side effects. In addition to the antidepressive effect, the pharmaceutical composition has other effects such as antianxiety effect, antimanic effect, inhibition of impulse, appetite control, reducing the desire to drink alcohol, and improving unidentified complaints such as neck stiffness.

A drug mixture of the pharmaceutical composition of the present invention and donepezil is effective on dementia and behavioral and psychological symptoms of dementia by once-daily administration. Administering a plurality of drugs to a patient of dementia several times daily involves much labor of the carer. Once-daily administration of the drug mixture before sleeping would improve dementia and behavioral and psychological symptoms of dementia with minimum side effects. Donepezil has a side effect to facilitate gastrointestinal motility. In contrast, the pharmaceutical composition of the present invention acts inhibitory on the gastrointestinal motility, and is therefore highly suitable for combination with donepezil since it would avoid the use of gastrointestinal agents in patients suffering from the gastrointestinal side effects of donepezil.

### DESCRIPTION OF EMBODIMENTS

All patents and literature references cited in this specification are hereby incorporated by reference in their entirety. The present invention contemplates methods of treatment of mental disorders in patients having or being susceptible to depression. In one embodiment, the present invention contemplates administration of an effective amount of 1,1-diphenyl-4-piperidine-1-ylbuthan-1-ol, which may hereinafter be referred to as “difenidol”, or its pharmaceutically acceptable salt, preferably in a pharmaceutically acceptable carrier or diluent. In another embodiment, another antidepressant, antianxiety, antipsychotic, antiepileptic, or hypnotic drug commonly used to treat mental disorders is administered in addition to the pharmaceutical composition of the present invention. Alternatively, an anti-dementia medication is administered as a combination medication with the pharmaceutical composition of the present invention to treat mental instability or insomnia associated with dementia. It should be understood that such a combination medication can also be further combined with a medication commonly used to treat mental disorders.

The compound used in the present invention is 1,1-diphenyl-4-piperidine-1-ylbuthan-1-ol, which is represented by the following formula:

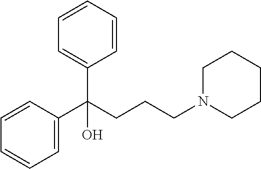

Difenidol is a known compound, and can be produced by those skilled in the art with a commonly used method. For example, it can be produced by reacting an organometallic compound that is obtained by reacting a halogenated butylpiperidine and metallic magnesium, with benzophenone (see U.S. Pat. No. 2,411,664, or GB68395). Difenidol may be in a free form or a pharmaceutically acceptable salt form. As a salt form, an acid addition salt is preferable. There are, for example, a salt of inorganic acid such as hydrochloric acid, sulfuric acid, nitric acid, phosphoric acid, hydrofluoric acid, and hydrobromic acid, and a salt of organic acid such as formic acid, acetic acid, lactic acid, adipic acid, citric acid, tartaric acid, fumaric acid, maleic acid, succinic acid, methanesulfonic acid, and benzenesulfonic acid. These salts can be produced by those skilled in the art with commonly used methods. Difenidol hydrochloride is an effective ingredient of a therapeutic drug for dizziness attributed to inner ear disorder, and is commercially available as a trade name Cephadol® or Mecalmin®.

The pharmaceutical composition of the present invention may contain, in addition to difenidol or a pharmaceutical salt thereof, other ingredients, for example, another antidepressant, antianxiety, antipsychotic, antiepileptic, hypnotic, or anti-dementia drug. Examples of other antidepressant drugs include, but are not limited to, tricyclic antidepressants such as amitriptyline, amoxapine, clomipramine, desipramine, doxepin, and imipramine; selective serotonin reuptake inhibitors such as citalopram, fluoxetine, fluvoxamine, paroxetine, and sertraline; monoamine oxidase (MAO) inhibitors such as phenelzine and tranylcypromine; and serotonin/noradrenaline reuptake inhibitors (SNRIs) such as milnacipran and duloxetine.

Examples of antianxiety drugs include benzodiazepine such as diazepam and alprazolam; and thienodiazepine such as etizolam and clotiazepam. Examples of antipsychotic drugs include anti-schizophrenic drugs such as chlorpromazine and olanzapine. Examples of antiepileptic drugs include, but are not limited to, phenobarbital, metharbital, ethotoin, phenytoin, clonazepam, and diazepam. In addition, examples of antidementia drugs include acetylcholinesterase inhibitors such as donepezil, rivastigmine, galantamine, tacrine, metrifonate, neostigmine and physostigmine.

Donepezil can be produced easily by a process disclosed, for example, in Japanese Patent Laid-Open No. 01-079151, Japanese Patent Nos. 2,578,475, 2,733,203 and 3,078,244 or U.S. Pat. No. 4,895,841. Donepezil hydrochloride is also available as a pharmaceutical preparation such as fine granule. Most of other acetylcholinesterase inhibitors are commercially available, and can be obtained from, for example, several chemical companies.

As used herein, the term “mental disorder” refers to those provided in the Diagnostic and Statistical Manual (DSM IV), American Psychological Association (APA). These mental disorders include, but are not limited to, affective disorders, neurotic disorders and unspecified depressive disorders. Examples of affective disorders include mood disorders, manic disorder, major depressive disorder and bipolar affective disorder. Mood disorders include, but are not limited to, depressive disorders, dysthymic disorder, bipolar disorders (I and II) and cyclothymic disorders. Likewise, examples of neurotic disorders include, but are not limited to, anxiety states, panic disorders, phobias, obsessive-compulsive disorder, post traumatic stress disorder, acute stress disorder, generalized anxiety disorder, attention deficit hyperactivity disorder, Tourette's Syndrome and hysteria. Other conditions include sleep disorders, including breathing-related sleep disorders.

The term “depression”, as used herein includes those described in DSM IV, including, but not limited to, mood disorders, depression clinically diagnosed by professionals, such as psychiatrists, psychotherapists, psychologists, and therapists, as well as depression which may not be clinically diagnosed by a mental health practitioner but may nevertheless still be severe and prolonged. By way of non-limiting examples, clinically diagnosed depression includes dementia, acute depression, schizophrenia, and other clinical depression disorders, classified in DSM IV.

For evaluation of the state of depression, for example, Hamilton's rating scale for depression (HAMD) is available. The HAMD was developed by Max Hamilton in 1960 for the purpose of measuring the severity of a patient diagnosed with depression (Hamilton, M., Journal of Neurology, Neurosurgery & Psychiatry, 23-56-62 (1960) and Hamilton, M., “Development of rating scale for primary depressive illness”, British Journal of Social and Clinical Psychology, 6: 278-296 (1967)). In view of its high usefulness, it is a representative scale of evaluation for depression, widely used in clinical researches and practices. The 17 item version is used for clinical trials of antidepressants in Japan, whereas the 21 item version and the 24 item version are used in Europe or other countries.

When the severity is calculated using Hamilton's rating scale for depression (HAMD), each item is evaluated with three grades of 0-2, or five grades of 0-4. For example, the case is diagnosed as severe where the point is 20 or higher and, as the point decreases, the symptom is diagnosed as being improved.

Dosing amounts and duration of the pharmaceutical composition of the present invention may vary according to the individual patient, the severity of the mental disorder or susceptibility to mental disorder and other factors which may affect the pharmaceutical kinetics of drug dosage and delivery. Usually, every 10 to 100 mg, preferably every 25 to 50 mg of diphenidol hydrochloride is orally administered to the patient 3 times a day.

The composition of the present invention may be administered in a number of pharmaceutically acceptable formulations including, for example, tablets, granules and caplets. Preferably, the formulations are administered orally for ease of convenience. As carriers for preparing such formulations, there may be employed, for example, commonly used excipients, binders, disintegrating agents, lubricants, coloring agents and corrigents, and, as necessary, there may also be used, for example, stabilizers, emulsifiers, absorption promoters, pH adjusting agents, antiseptics, antioxidants, fillers, moisturizers, surface activators, dispersing agents, buffers, preservatives, and dissolving aids. The composition of the present invention can be formulated into a pharmaceutical preparation by a conventional method, by mixing components that are commonly used as materials for pharmaceutical preparations.

In the case of combining the pharmaceutical composition of the present invention with another drug, the respective active ingredients to be combined may be directly administered in their effective doses simultaneously or at different times. The active ingredients to be combined may be directly mixed together to make a pharmaceutical preparation, or may be separately formulated to some extent initially and then mixed together to make a pharmaceutical preparation, and the pharmaceutical preparation thus obtained may be administered in an effective dose. A pharmaceutical product that is prepared in the form of a single drug containing a mixture of a plurality of kinds of active ingredients having similar or different effects will be referred to as “a drug mixture.” A plurality of active ingredients may be made into separate pharmaceutical drugs, which may be provided in a kit. The kit contains a package insert providing information on dosage and administration, including the dosage per day, the frequency of administration, the route of administration and so on, for each drug.

In one embodiment, the pharmaceutical composition of the present invention can be administered alone, after discontinuation of treatment with another antidepressant, antianxiety, antipsychotic, antiepileptic, or hypnotic drug as a combination partner. Most of such other drugs cause withdrawal symptoms such as anxiety, restlessness, and anger, upon discontinuation thereof. As a result, it is often difficult to discontinue the administration of them. Continued administration of unnecessary drugs having side effects is a disadvantage, which would make women hesitate to be pregnant. In such a situation, the pharmaceutical composition of the present invention can be administered to relieve the withdrawal symptoms significantly.

In a preferred embodiment of the present invention, the mental disorder as a therapeutic target is fibromyalgia, chronic fatigue syndrome (CFS), or depression with pain, and the administration of the pharmaceutical composition of the present invention is capable of reducing the pain caused by these diseases.

Fibromyalgia is a clinical syndrome of widespread pain, fatigue, poor sleep, and chronic aching in multiple areas of the musculoskeletal system characterized by reproducible increased tenderness at specific sites (Bradley et al., Rheum Dis Clin North Am 1999, 25 (1): 56; and Pellegrino et al., Arch Phys Med Rehabil, 1999, 70 : 61). Pain in patients with fibromyalgia diffusely radiates from the axial skeleton over large areas of the body, predominantly involving muscles, and is described as exhausting, burning, miserable, or unbearable. The 1990 American College of Rheumatology (ACR) classification criteria for the diagnosis of fibromyalgia are the presence of widespread pain for more than three months and pain, not just tenderness, that can be elicited by manual pressure of approximately 4 kg/cm² at 11 or more defined tender points (Bradley et al., Rheum Dis Clin North Am 1999, 25 (1) : 56).

The pain of fibromyalgia is estimated to develop through a route different from that of so-called general pain. Namely, in the route of general pain, an inflammation and stimulation is transferred from each part of the body to pain receptors, and the stimuli go to the brain via the spinal cord to cause the feeling of pain. However, in the case of fibromyalgia, it is said that anxiety to pain causes several mental symptoms, which leads to a further pain-inducing factor. There are many disputes about disease pathogenesis of fibromyalgia, but no definitive cause has been eclucidated yet. It is reported so far that fibromyalgia relates to a defect of the central nervous system, inheritance, and psychological and social factors.

A disease that shares many clinical features with fibromyalgia is Chronic Fatigue Syndrome (CFS), which is a chronic, debilitating condition characterized by fatigue, cognitive problems, and various pains and other symptoms. The lives of patients are often frequently disrupted and as yet there is no effective therapy for this condition. Most of the patients are female, and the first onset of both diseases is usually in their thirties or forties. More than 80% of both clinical categories include fatigue, muscular pain, joint pain, repeated headache, and sleep disorder.

There are two criteria generally used for diagnosis of CFS. The criteria outlined by the US Centers for Disease Control and Prevention include medically unexplained fatigue of at least 6 months duration that is of new onset, not a result of ongoing exertion and not alleviated by rest. In addition, the diagnosis involves the determination of at least four additional symptoms including: tender lymph nodes, memory impairment, muscle pain, joint pain, headaches, un-refreshing sleep, and post-exertional malaise lasting for more than 24 hours. The Oxford criteria include severe disabling fatigue of at least 6 months duration that affects both physical and mental functioning with the fatigue being present for more than 50% of the time and also other symptoms including myalgia and sleep disturbances.

The pharmaceutical composition of the present invention can relieve various symptoms of fibromyalgia and CFS, such as, in particular, pain. The term “pain” in the present specification includes allodynia (painful response to a stimulus that is not usually painful) and hyperalgesia (exaggerated response to a stimulus that is usually only mildly painful); a series of regional pains, such as noncardiac chest pain, dyspepsia, headache, abdominal cramping (irritable bowel syndrome), temporomandibular pain and chronic pelvic pain; neck stiffness; and chronic aching in multiple areas of the musculoskeletal system.

The dosage form and dose of the pharmaceutical composition of the present invention for pain relief are not particularly different from those for improving depression, but can be preferably administered by controlled release means or by delivery devices that are well known to those of ordinary skill in the art. Examples thereof include, but are not limited to, those described in U.S. Pat. Nos.: 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide slow or controlled-release of one or more active ingredients using, for example, hydroxypropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled-release.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e. g., adverse) effects.

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

Examples which will be shown below are mere exemplifications and they just intend to illustrate the present invention in detail together with the above-mentioned preferred embodiments and do not limit the present invention thereto. Any person skilled in the art is able to modify the present invention without departing from the spirit of the present invention and the modification as such is also included within the scope of the present invention.

### EXAMPLES

Case 1: Attention Deficit Hyperactivity Disorder (ADHD)

Patient was a 37-year-old woman, who had often shown violent behaviors. Quarreling with a bad-mannered person on the road and performing violent acts such as hitting had occurred once or twice a week. Antidepressant, antiepileptic, and antianxiety drugs were all ineffective to her, but administration of 75 mg/day Cephadol® resulted in reduction of impulsivity on day two, and disappearance of the impulsivity on day seven. When she discontinued the medication with 75 mg/day Cephadol®, the symptom returned to the previous state. The effect of Cephadol® appeared in about two days, and the symptom returned to the original state in 5 days after discontinuation of the drug. There were no side effects.

Case 2: Refractory Depression

Patient was a 42-year-old woman, who was unresponsive to any combination of SSRIs, SNRIs, tricyclic antidepressants, and antianxiety agents in their respective maximum doses. The score of HAMD was 23 points. When the medication was changed to Cephadol® 75 mg/day, the patient noted a significant improvement in her depressive symptom. Increasing the dose of Cephadol® to 150 mg/day improved the score of HAMD to 3 points. The improvement lasted even after other antidepressants were removed. Even 3 months later, the score of HAMD was still 3 points. When the dose of Cephadol® was reduced to 75 mg/day, the score of HAMD became 13 points after one week. There were no side effects.

Case 3: Manic-Depressive Illness

Patient was a 42-year-old man, who had continuously been in a manic state of bipolar I disorder for over one month. An antiepileptic drug, or 1,200 mg of Limas®, was ineffective. Antipsychotic drugs were not usable due to significant side effects. The change of the medication to Cephadol® 150 mg/day led to a significant amelioration of the disease. When he discontinued the administration of Cephadol®, the manic state appeared after 5 days. There were no side effects.

Case 4: Alzheimer-Type Dementia

Patient was a 76-year-old man with dementia, who was difficult to treat with donepezil hydrochloride due to the excitatory side effect. Additional administration of 50 mg Cephadol® before sleeping reduced the excitation. This allowed treatment with donepezil hydrochloride, which resulted in a significant improvement of the dementia. There were no side effects.

Case 5: Effect on Behavioral and Psychological Symptoms of Dementia

Patient was an 82-year-old woman with dementia, who was irritable and easily excited, and had persecutory delusion. Administration to the patient of 100 mg/day Cephadol® significantly improved mental stability, and prevented her nuisance behaviors to other people caused by the persecutory delusion. There were no side effects.

Case 6: Alcoholism

Patient was a 45-year-old male office worker, who was drinking 1 to 2 liters of beer and 3 to 5 glasses of distilled spirit or whisky with water every day. He was drinking alcohol in daytime on Sundays, and suffering from a complication of liver dysfunction. Cyanamide and antidepressants had been used without any efficacy. Administration to the patient of 150 mg/day Cephadol® eliminated his impulse to drink alcohol on the next day, and also eliminated his restlessness and anxiety at the same time. The patient continued to take the drug for three months without any emergence of overdrinking and without any side effects.

Case 7: Bulimia (1)

Patient was a 26-year-old woman, who had been suffering from bulimia since the age of 18. Antidepressant, antianxiety, and other drugs were all ineffective. Administration to the patient of 150 mg/day Cephadol® significantly improved overeating, which had previously occurred at a frequency of two to three times a day. The effect of Cephadol® appeared in about two days. There were no side effects.

Case 8: Bulimia (2)

Patient was a 31-year-old woman, who had been suffering from bulimia since the age of 18. The bulimia persisted even after her marriage and childbearing. She took 40 mg Prozac® by private import, which slightly improved the bulimia. Overeating and emesis, which had previously occurred at a frequency of two to three times a day, was reduced to once daily, but still existed. Administration to the patient of 150 mg/day Cephadol® stopped her overeating. The effect remains for more than two months without any side effects.

Case 9: Depressed Symptoms

Patient was a 32-year-old male office worker with depression, who had been treated with 40 mg of Paxil® (paroxetine) without any improvement. Administration to the patient of 75 mg/day Cephadol® reduced the HAMD score from 20 points to 7 points after 3 days. There were side effects.

Case 10: Panic Disorder and Multiple Personality Disorder

Patient was a 35-year-old single woman, who had been treated at several psychiatric hospitals. She had previously consulted 7 psychiatrists, and had taken almost all of existing mental drugs, without any improvement. The inventor treated the patient with 75 mg/day Cephadol® for one week, and then increased the dose of Cephadol® to 150 mg/day, which resulted in a significant improvement of panic disorder. The transformation of personality, which had previously occurred at a frequency of 5 to 10 times a month, was also improved. She reports that the medicine is best to calm her mind, and easier to take than any other drugs that she has ever taken. It was found that Cephadol® has a strong antianxiety effect.

Case 11: Insomnia in an Elderly Person

A 78-year-old man had had no history of insomnia or depression before he retired from his company at mandatory retirement age. Insomnia occurred when he was over 70 years old. Being seriously distressed by insomnia, he had been prescribed hypnotics at two or three mental clinics. The hypnotics had made him easily fall down when going to the bathroom at night, and made him feel half awake in the morning, with memory impairment. The patient was started on 50 mg Cephadol® twice a day (after dinner and before sleeping). As a result, he became able to sleep well, and his hypnotics were reduced in dose by half. This made him free from staggering gait on awaking at night, and made him feel well on the next morning, without memory impairment. There were no side effects.

There are many cases of insomnia in elderly persons. The elderly patients taking hypnotics are often unsteady on their legs upon awaking at night, which causes an accident. In contrast, Cephadol® leads to a safe and complete cure, without causing a stagger. In contrast to Cephadol®, which pharmacologically acts in a blood vessel, most hypnotics act on outside of the blood vessel, parenchymal brain, and therefore tend to cause side effects. In addition, dementia in elderly persons is often accompanied by a disorder of sleeping rhythms and insomnia. Since antidementia agents act excitatory on the brain, they often cause insomnia. Cephadol® improves such insomnia effectively, and also ameliorates various portions of the brain. Thus, it is highly suitable as a supplementary agent to antidementia drugs.

Case 12: Schizophrenia

A 45-year-old man had been diagnosed with schizophrenia at the age of 20 and had received antipsychotic drugs. In spite of the treatment with newest antipsychotic drugs, the dullness of thinking, depression, and obesity of the patient were not improved. Administering 150 mg/day Cephadol® to the patient reduced his appetite and body weight. It also improved his subjective and objective thinking speed and depression, thus resulting in improvement in his quality of life. There were no side effects, nor relapse of schizophrenia.

Case 13: Withdrawal Symptoms

A 35-year-old male office worker had been on 10 mg Paxil®, 2 mg Rohypnol® and 2 mg Depas® before sleeping. Although he had tried to reduce the doses of the drugs, emergence of withdrawal syndrome had hampered the reduction of the drugs. He started to take Cephadol®, 25 mg after dinner and 50 mg before sleeping. It made him able to be free from the other drugs in two weeks. The dose of Cephadol® was then reduced from 50 mg to 25 mg, and to 0 mg, which resulted in discontinuation of all the drugs. There were no side effects.

Case 14: Stiff Neck

Stiff neck is often observed as a subjective symptom, and is one of the most serious complaints in the daily life. Seven subjects having stiff neck were given two 25 mg tablets of Cephadol® two times a day (after dinner and before sleeping). All the subjects noted a disappearance or a significant improvement of the symptom after two days.

Case 15: Fibromyalgia (1)

A 56-year-old woman (housewife) had been diagnosed with fibromyalgia by an expert doctor and had been treated for the subsequent three years with a variety of medicines such as non-steroidal antiinflammatory drugs, antianxiety drugs, a tricyclic antidepressant Triptanol, SSRIs, and SNRIs at their respective maximum doses without any improvement. She then visited the inventor's clinic.

When the inventor started the treatment with 75 mg/day Cephadol®, systemic pain of the patient was improved in two days. One week later, the dosage of Cephadol® was increased to 150 mg/day, which resulted in complete elimination of arthralgia. Pain at 18 tender points including low back pain was totally improved. She felt that joints of the whole body became softer, and objectively, the mobility of large joints increased. As a result, she became able to perform usual household work, go outside, and further make a trip.

Case 16: Fibromyalgia (2)

A 34-year-old woman (housewife) had been treated for 6 years in the inventor's clinic. There were persistent systemic malaise, lassitude, depressed mood, sleeplessness, neck stiffness and systemic pain. Her score of depression on Hamilton D scale was 25 to 28. No hematological abnormality was detected. As a result of overall judgment based on examinations including a tender point examination, she was diagnosed as having fibromyalgia.

She was unresponsive to tricyclic or tetracyclic antidepressants, SSRIs, SNRIs, and Moclobemide. Then, she continued the treatment with 60 mg/day fluoxetine. Additional administration of 100 mg/day Cephadol® for one week actually reduced neck stiffness and systemic pain. Before the Cephadol® administration, she had been unable to perform any household work or go outside without an attendant, but after two weeks of the Cephadol® administration, her activity of daily living improved drastically. The state of depression was also improved to score 16 of Hamilton D scale. It was concluded that the onset of her depression was due to the systemic pain, and the depression was improved by the drastic improvement of the systemic pain.

Case 17: Fibromyalgia (3)

A 58-year-old woman (housewife) was diagnosed with fibromyalgia at another hospital. Being unresponsive to prescribed medicines, she was instructed to do gentle stretching exercise and massage, as well as thermotherapy on affected areas. However, her arthralgia was so terrible that she could not perform stretching exercise or other directions. She had pain at all the 18 tender points of the whole body, a jaw abnormality, and a strong pain at both knee joints, which made her unable to go to hospital on foot.

Administration of 75 mg/day Cephadol® improved the pain at both knee joints, and eliminated the systemic pain. When the dosage of Cephadol® was increased to 125 mg/day, the pain at both knee joints also disappeared. Reduction of the dosage of Cephadol® to 75 mg/day caused a recurrence of the pain at both knee joints on the next day. Re-increasing the dose of Cephadol® to 125 mg/day eliminated the pain at both knee joints on the next day, and enabled her usual daily life and social life. She felt that joints of the whole body were softer, and objectively, the mobility of large joints increased.

Case 18: Fibromyalgia (4)

A 42-year-old woman (housewife) is a patient with depression, having received regular medical treatment since seven years ago. She had a long lasting slight fever, chronic headache, and a strong malaise. However, a diagnosis with autoimmune disease or inflammatory disease was negative from her blood test. Due to back pain and fingertip pain, she was unable to make household work, and it was painful for her to hold even a light object by hand. She had pain at 15 points among the 18 tender points of the whole body, and was diagnosed with fibromyalgia. She was unresponsive to any kind of antidepressants, among which, Milnaciplan, one of SNRIs, rather deteriorated her back pain. She had to stay in bed all day long, and such a condition lasted for the long term. When she commenced on 75 mg/day Cephadol®, a significant improvement in malaise was noted. Increase of the dose of Cephadol® to 125 mg/day improved the back pain and fingertip pain, and enabled her usual household work. She felt that joints of the whole body were softer, and objectively, the mobility of large joints increased.

1-9. (canceled)

10. A method of treating a mental disorder comprising administering to a patient in need thereof, an effective amount of a pharmaceutical composition comprising 1,1-diphenyl-4-piperidine-1-ylbuthan-1-ol or a pharmaceutically acceptable salt thereof.

11. The method of claim 10, wherein said mental disorder is depression, bipolar disorder, anxiety disorder, impulsive disorder, bulimia, panic disorder, social anxiety disorder, insomnia, attention deficit hyperactivity disorder (ADHD), schizophrenia, dementia, personality disorder, alcoholism, dissociative disorder, sleep apnea syndrome, or fibromyalgia.

12. The method of claim 10, wherein said pharmaceutical composition comprises difenidol hydrochloride.

13. The method of claim 10, wherein said pharmaceutical composition is administered to the patient in combination with another antidepressant, antianxiety, antipsychotic, antiepileptic, or hypnotic drug.

14. The method of claim 13, wherein said pharmaceutical composition is administered simultaneously with or separately from the other antidepressant, antianxiety, antipsychotic, antiepileptic, or hypnotic drug.

15. The method of claim 10, wherein said mental disorder is fibromyalgia, chronic fatigue syndrome (CFS), or depression with pain, the method reducing the pain caused by said mental disorder.

16. A method of treating a mental disorder, comprising administering an effective amount of a pharmaceutical composition comprising 1,1-diphenyl-4-piperidine-1-ylbuthan-1-ol or a pharmaceutically acceptable salt thereof to a patient in need of treatment of insomnia or delirium associated with dementia, or behavioral and psychological symptoms of dementia such as persecutory delusion, prowl, and violent behavior.

17. The method of claim 16, wherein said pharmaceutical composition is prepared as a drug mixture with donepezil hydrochloride.

18. The method of claim 13, wherein said pharmaceutical composition is administered alone after discontinuation of treatment with the other antidepressant, antianxiety, antipsychotic, antiepileptic, or hypnotic drug.

19. The method of claim 10, wherein said mental disorder is impulsive disorder, bulimia, panic disorder, social anxiety disorder, insomnia, attention deficit hyperactivity disorder (ADHD), sleep apnea syndrome, or fibromyalgia.

20. The method of claim 10, wherein said mental disorder is impulsive disorder.

21. The method of claim 10, wherein said mental disorder is bulimia.

22. The method of claim 10, wherein said mental disorder is panic disorder.

23. The method of claim 10, wherein said mental disorder is attention deficit hyperactivity disorder (ADHD).

24. The method of claim 15, wherein said mental disorder is fibromyalgia.
